(19) United States
(12) Patent Application Publication
(10) Pub. No.: US 2023/0112547 A1
(43) Pub. Date: Apr. 13, 2023

(54) CONTACTLESS HEALTHCARE SCREENING

(71) Applicant: EXA HEALTH, INC., Los Altos, CA (US)

(72) Inventors: Siddarth Satish, Redwood City, CA (US); Steven Scherf, Oakland, CA (US); Griffeth Tully, Menlo Park, CA (US); Charles Peterson Carroll, Berkeley, CA (US); Thang Sy, Menlo Park, CA (US)

(21) Appl. No.: 17/278,570

(22) PCT Filed: Mar. 19, 2021
(86) PCT No.: PCT/US21/23315

§ 371 Date: Mar. 22, 2021

Related U.S. Application Data
(60) Provisional application No. 62/992,883, filed on Mar. 20, 2020.

(51) Int. Cl. G16H 10/20

(52) U.S. Cl. CPC G16H 10/20; G06K 19/06037

(57) ABSTRACT

One or more devices may be configured to provide or otherwise facilitate contactless healthcare screening. A mobile app may be installed on a device of a patient, and the mobile app may cause the device to guide the patient through a healthcare screening, for example, including administration of a healthcare screening questionnaire, without risk of making physical contact with the healthcare worker. The same mobile app or another mobile app may be installed on a device of a healthcare worker, and the mobile app may cause the device to guide the healthcare worker in administering the healthcare screening to the patient, also without risk of making physical contact with the patient.

### RELATED APPLICATION

This application is a national phase entry of PCT/US21/23315, titled “CONTACTLESS HEALTHCARE SCREENING” and filed Mar. 19, 2021, which claims the priority benefit of U.S. Provisional Patent Application No. 62/992,883, titled “CONTACTLESS HEALTHCARE SCREENING” and filed Mar. 20, 2020, which is incorporated herein by reference in its entirety.

### TECHNICAL FIELD

The subject matter disclosed herein generally relates to the technical field of special-purpose machines that facilitate healthcare screening, including software-configured computerized variants of such special-purpose machines and improvements to such variants, and to the technologies by which such special-purpose machines become improved compared to other special-purpose machines that facilitate healthcare screening. Specifically, the present disclosure addresses systems and methods to facilitate contactless healthcare screening.

### BACKGROUND

Healthcare screening typically is performed by a front-line healthcare worker (e.g., a triage nurse) administering a questionnaire to a newly arrived patient seeking healthcare services. Some or all of the questionnaire may be administered to the patient while the front-line healthcare worker and the patient are effectively in contact with each other or otherwise in close enough proximity to risk transmission of pathogens from one to the other.

### BRIEF DESCRIPTION OF THE DRAWINGS

Some embodiments are illustrated by way of example and not limitation in the figures of the accompanying drawings.

FIGS. 1-4 are screenshots describing an example use case in which contactless healthcare screening may be utilized, according to some example embodiments.

FIGS. 5-13 are screenshots describing an overview of contactless healthcare screening, according some example embodiments.

FIGS. 14 and 15 are screenshots illustrating some screens of an example graphical user interface (GUI) for contactless healthcare screening, according to some example embodiments.

FIGS. 16-26 are screenshots illustrating some screens of an example GUI for contactless healthcare screening, as presented by a device of healthcare worker to facilitate administration of a healthcare screening for a patient, according to some example embodiments.

FIGS. 27-48 are screenshots illustrating some screens of an example GUI for contactless healthcare screening, as presented by a device of a patient to facilitate participation in a healthcare screening of the patient, according to some example embodiments.

FIG. 49 is a flowchart illustrating operations of a first mobile device of a patient and a second mobile device of a healthcare worker, in performing a method of contactless healthcare screening, according to some example embodiments.

FIG. 50 is a block diagram illustrating components of a machine according to some example embodiments, able to read instructions from a machine-readable medium and perform any one or more of the methodologies discussed herein.

### DETAILED DESCRIPTION

Example methods (e.g., algorithms) facilitate contactless healthcare screening, and example systems (e.g., special-purpose machines configured by special-purpose software) are configured to facilitate contactless healthcare screening. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will be evident to one skilled in the art, however, that the present subject matter may be practiced without these specific details.

One or more devices (e.g., a mobile device, such as a smartphone, or a computer) may be configured (e.g., by suitable hardware, software, or both) to provide or otherwise facilitate contactless healthcare screening. A mobile app may be installed on a device of a patient, and the mobile app may cause the device to guide the patient through a healthcare screening (e.g., including administration of a healthcare screening questionnaire) without risk of making physical contact with the healthcare worker. The same mobile app or another mobile app may be installed on a device of a healthcare worker, and the mobile app may cause the device to guide the healthcare worker in administering the healthcare screening to the patient, also without risk of making physical contact with the patient. In some example embodiments, the same mobile app is installed on both devices, and different user inputs to the mobile app cause the mobile app to operate in a patient mode (e.g., with a patient interface, such as a patient graphical user interface (GUI)) on the device of the patient or otherwise operate in a healthcare worker mode (e.g., with a healthcare worker interface, such as a healthcare worker GUI) on the device of the healthcare worker.

For example, both devices may download, install, and execute a mobile app specifically configured for contactless COVID-19 screening and triage. Such a mobile app may be deployed at permanent or temporary medical facilities on the front lines of providing healthcare to people potentially or actually exposed to the virus that causes COVID-19. Healthcare workers are increasingly receiving symptomatic patients who have bypassed telehealth visits or telehealth chatbots, or who may not have had access to such telehealth resources. Such patients may present to a hospital or clinic with no appointment. It may be beneficial to pathogenically isolate healthcare workers from coming into contact with sick patients.

According to the systems and methods discussed herein, when a patient arrives and presents himself or herself to an emergency room (ER) or other healthcare facility, the patient downloads a mobile app by pointing the camera of a device of the patient (e.g., his or her smartphone camera) at a quick response (QR) code, which may be prominently displayed on a sign or display screen. Next, the downloaded mobile app guides the patient (e.g., via GUI prompts) through a healthcare screening questionnaire by which the patient fills out an assessment of symptoms and risk factors based on official (e.g., Centers for Disease Control (CDC)) guidelines, and the mobile app then encodes a summary of the patient's results within another QR code (e.g., an output QR code). The mobile app prompts the patient to show this resultant QR code to a healthcare worker. A physical barrier (e.g., automotive window glass), suitable distance (e.g., 6-10 feet), or both, may separate the patient from the healthcare worker and thus provide full or partial pathogenic isolation of the healthcare worker from the patient.

The healthcare worker uses the mobile app (e.g., operating in healthcare worker mode) on his or her device to scan the patient's presented QR code and thereby obtain the summary of the patient's questionnaire results. For example, the patient's QR code may be scanned by a healthcare worker through a fully rolled up car window at a drive-through medical testing facility to obtain and view the patient's symptoms and risk factors on the healthcare worker's own device. As another example, the patient's QR code may be scanned through a transparent barrier, such as a rigid clear plastic panel or a flexible clear plastic tent wall. In some example embodiments, the healthcare worker can additionally make a phone call to the patient, who is still in their car, to facilitate further healthcare screening while continuing to limit the exposure risk for the healthcare worker. The mobile app, in certain example embodiments, may support private and confidential person-to-person messaging, voice calls, video calls, or any suitable combination thereof, for the healthcare worker and the patient to communicate with each other.

In various example embodiments, some or all of the functionality described above for the mobile app is also available via a web interface hosted by a web server. Accordingly, the systems and methods discussed herein may be flexibly deployed in various healthcare settings, such that doctor visits, urgent care, and emergency room treatment can each respectively provide pathogenic isolation of the relevant healthcare worker (e.g., a doctor, a nurse, or a medical technician) while administering a healthcare screening to a patient.

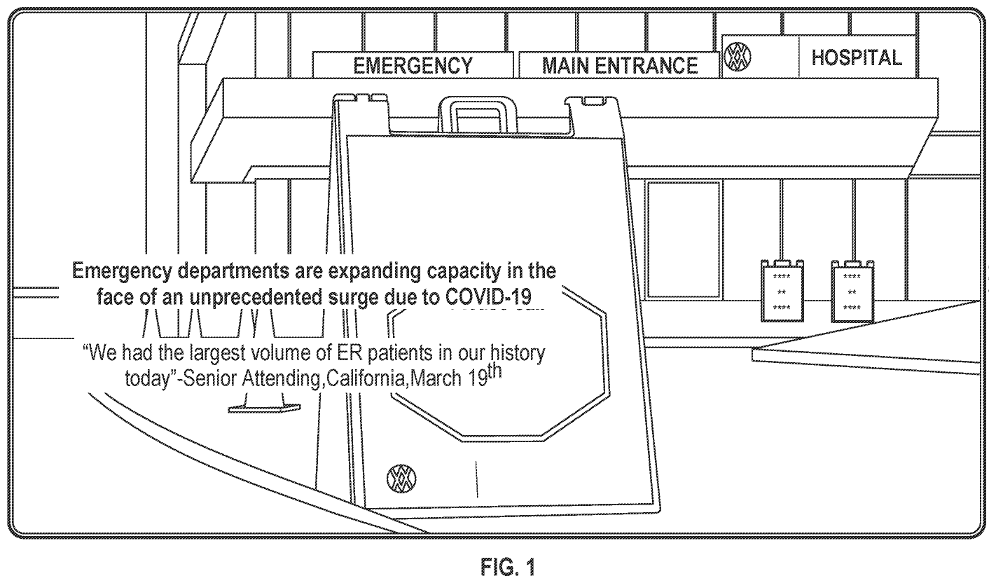

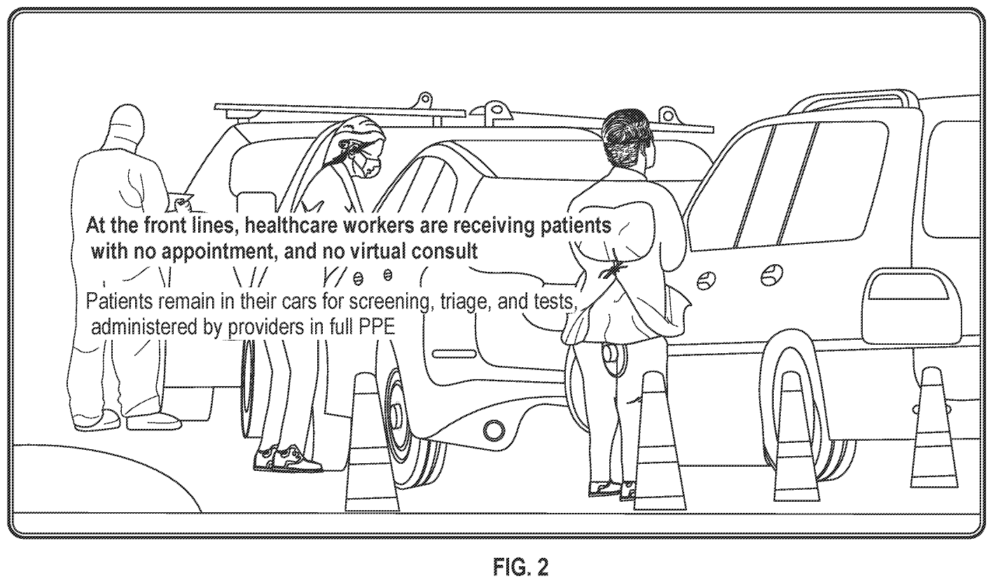

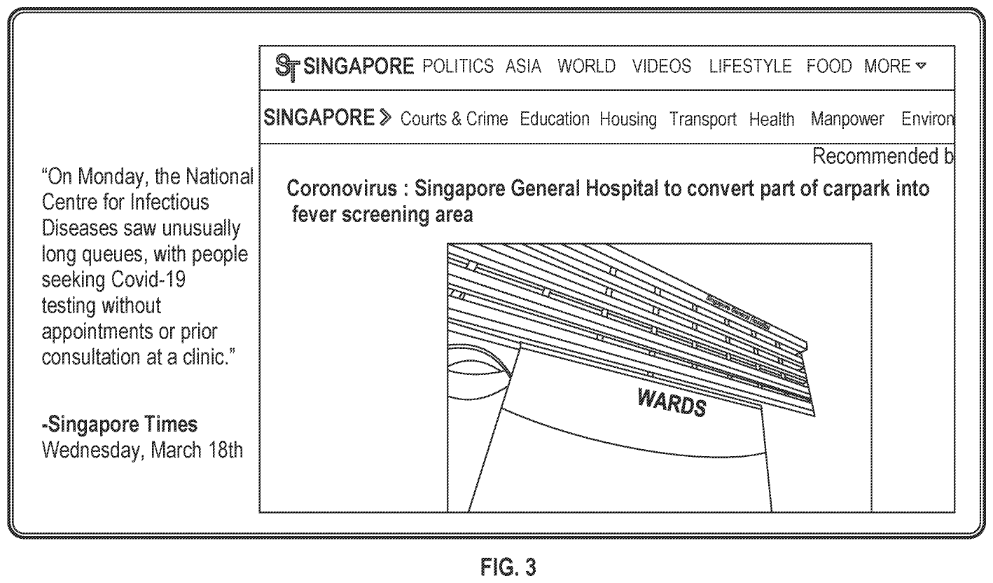

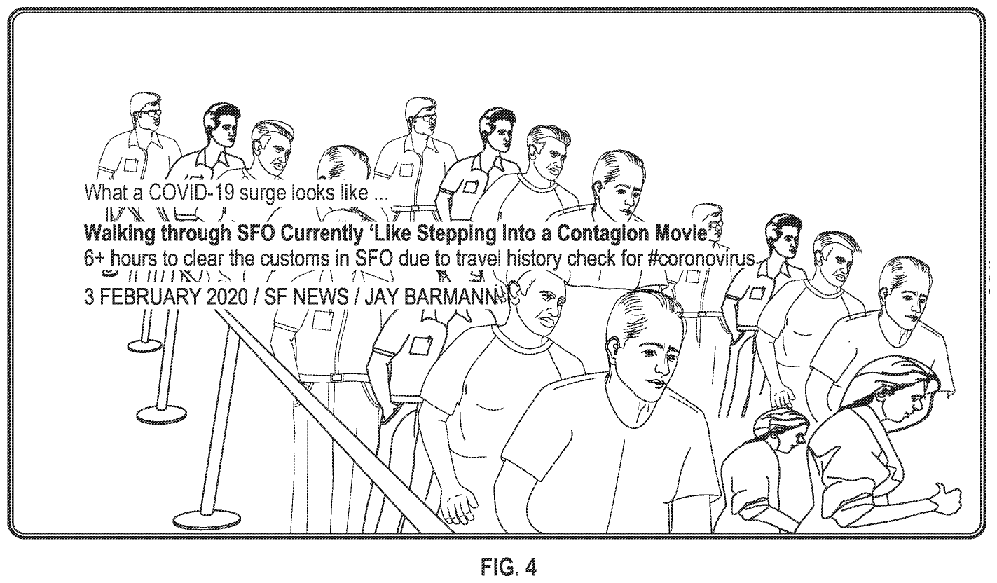

FIGS. 1-4 are screenshots describing an example use case in which contactless healthcare screening may be utilized, according to some example embodiments.

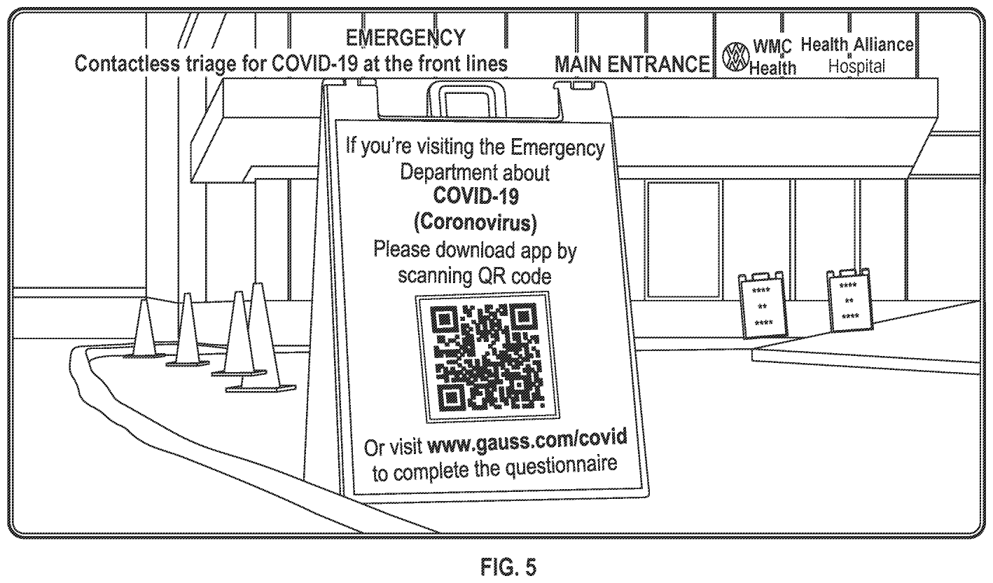

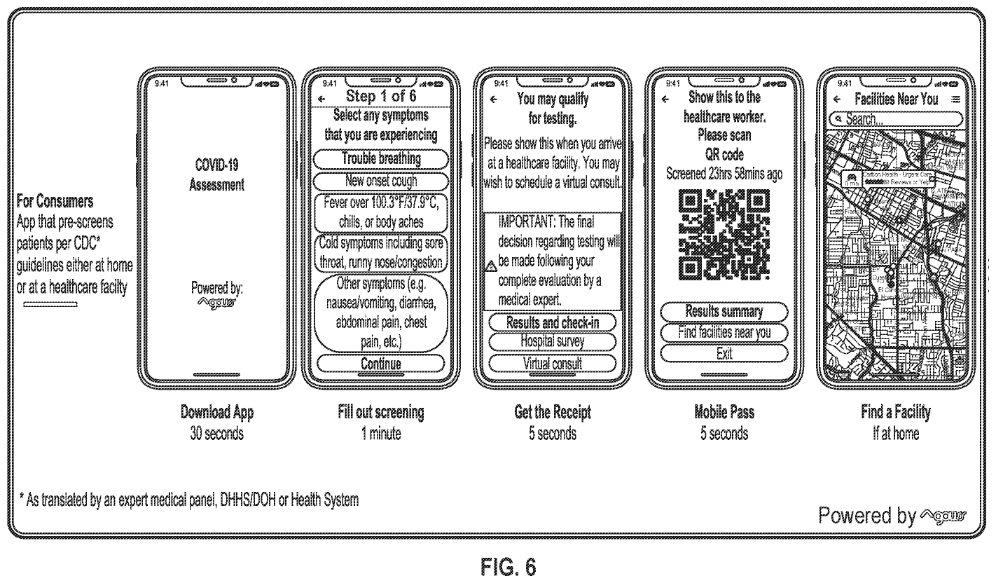

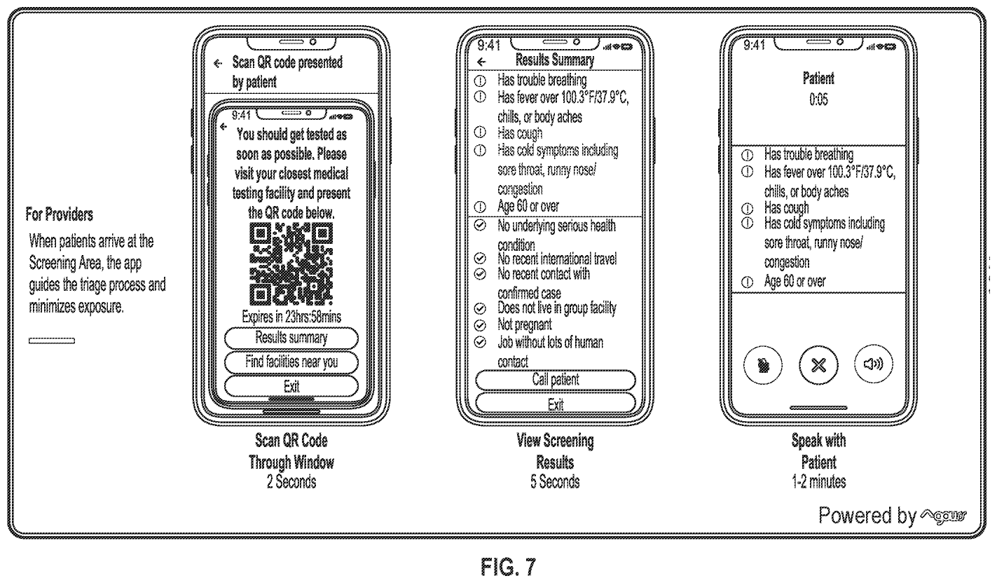

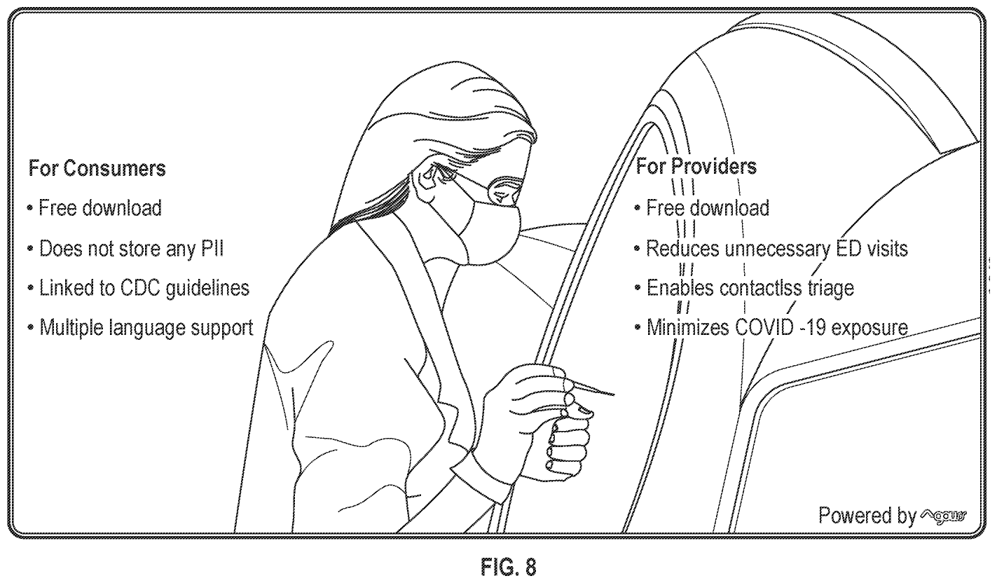

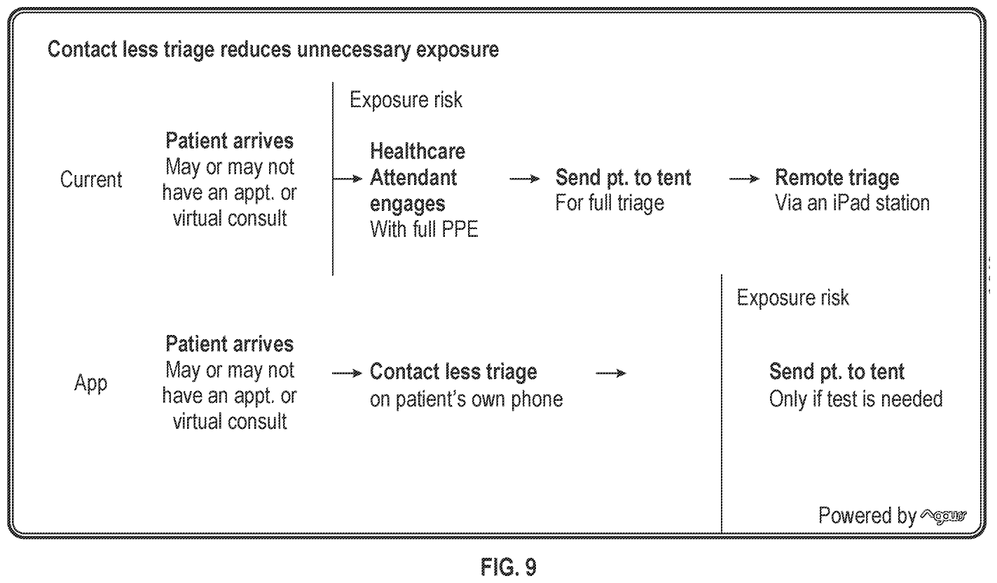

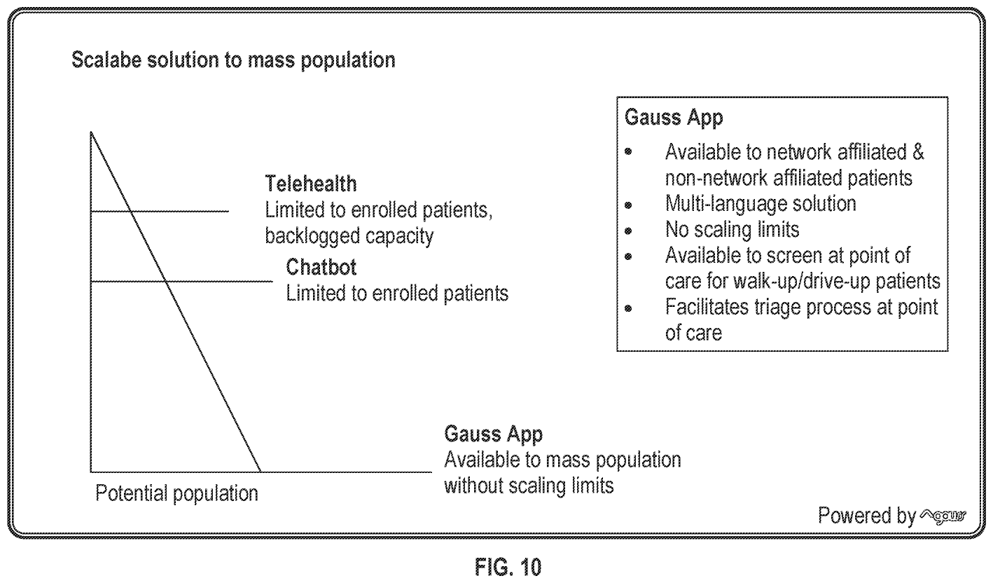

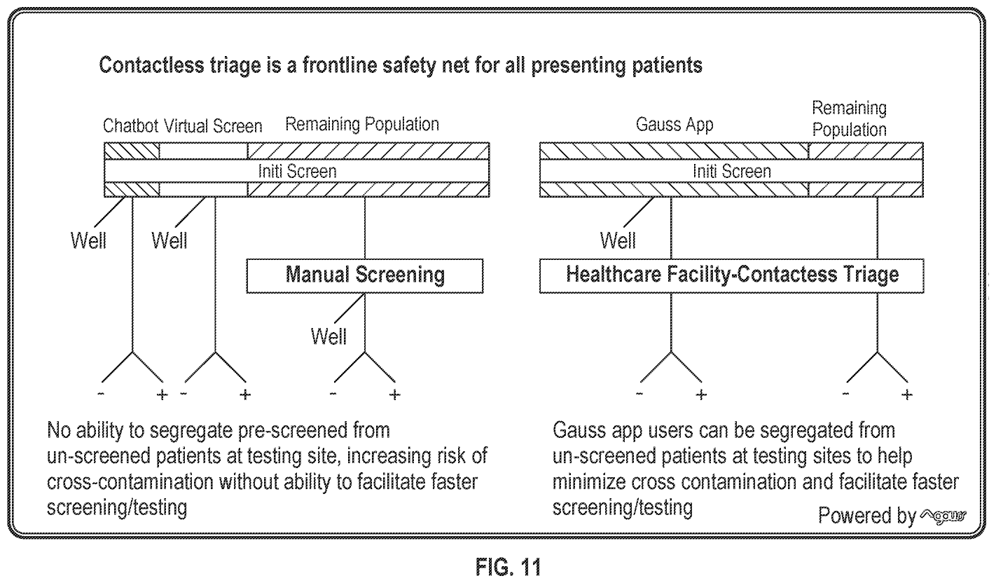

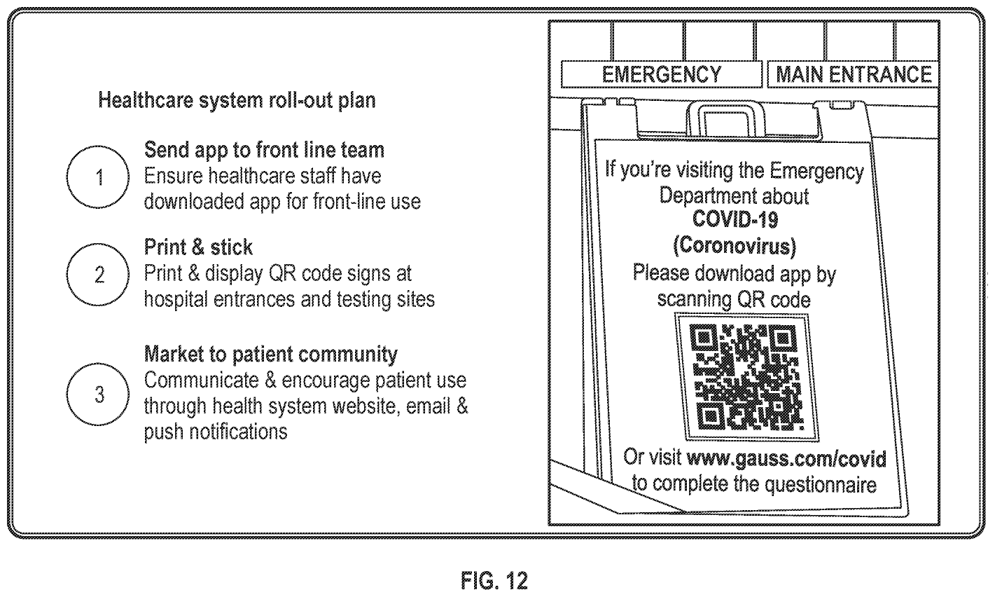

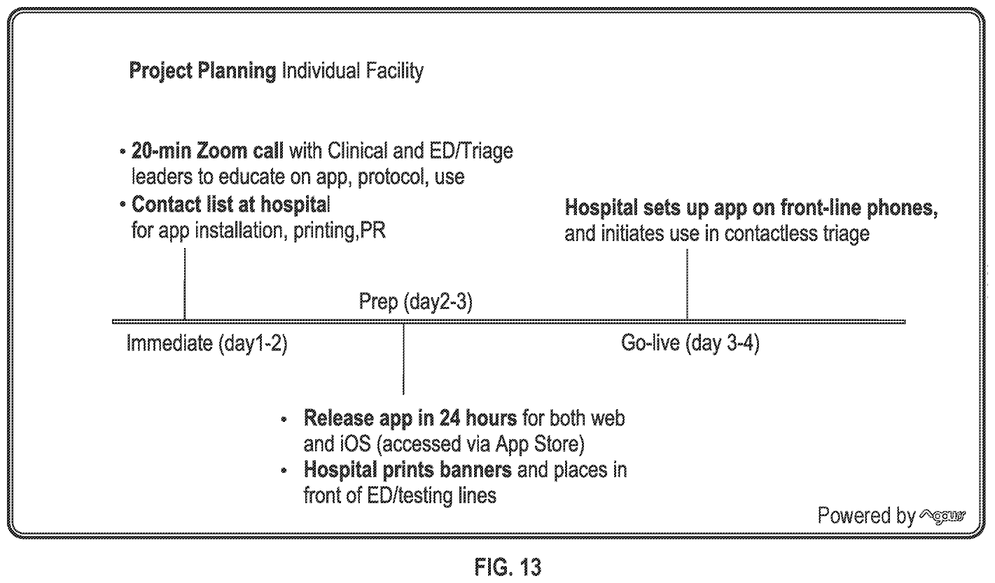

FIGS. 5-13 are screenshots describing an overview of contactless healthcare screening, according some example embodiments.

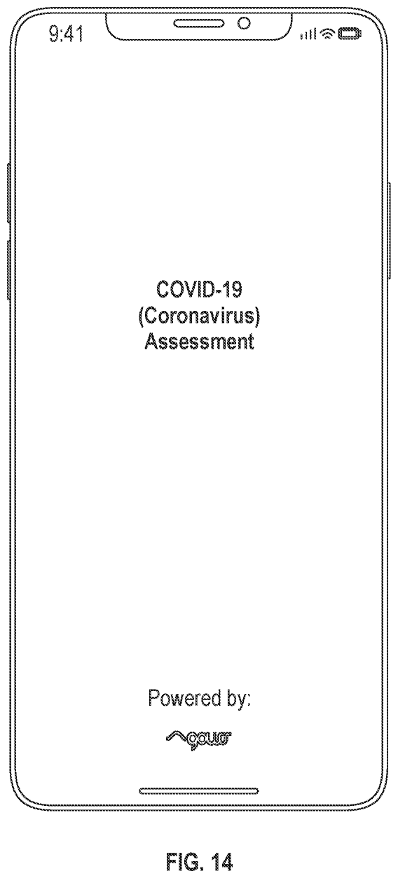

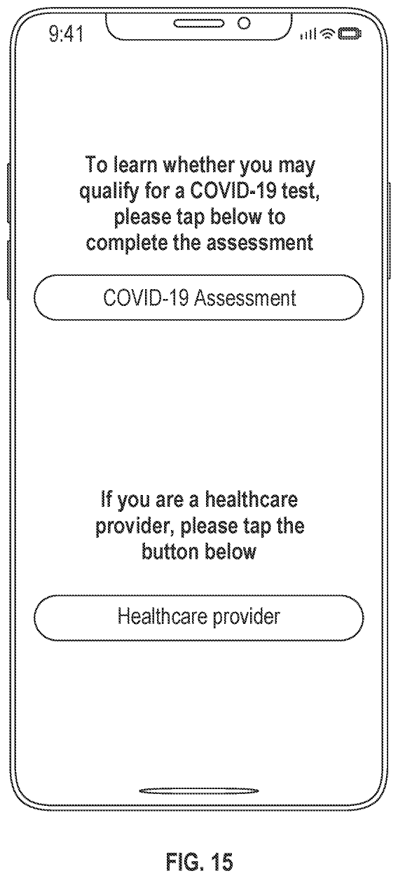

FIGS. 14 and 15 are screenshots illustrating some screens of an example graphical user interface (GUI) for contactless healthcare screening, according to some example embodiments.

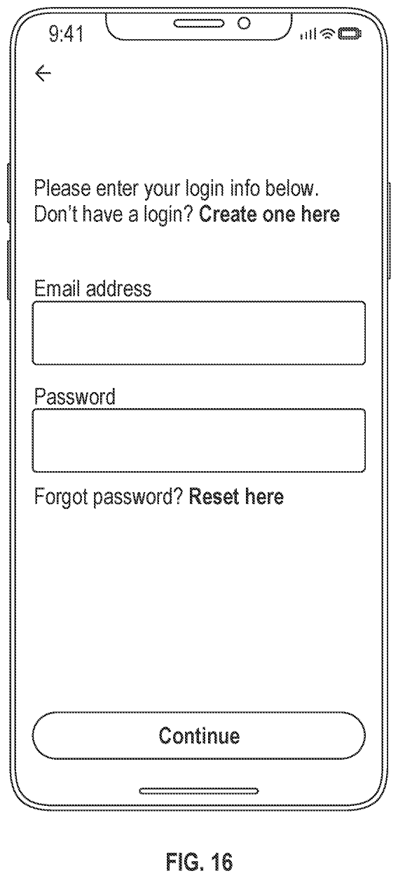

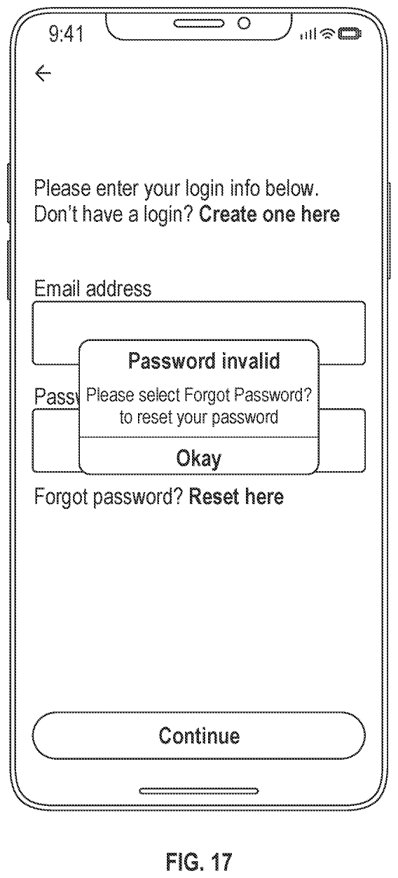

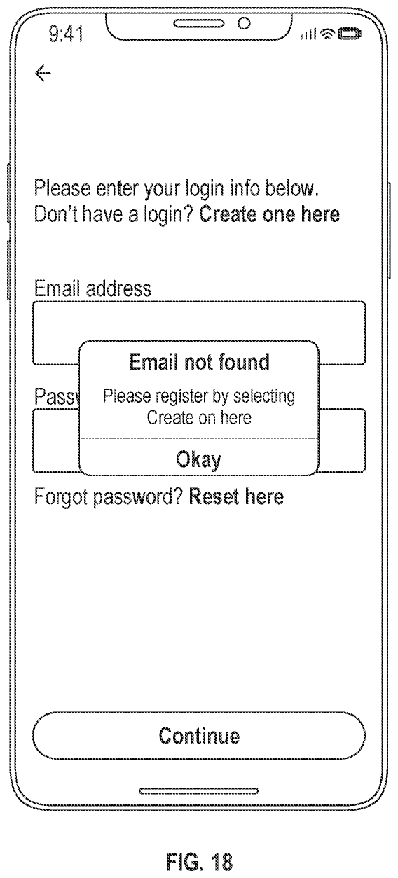

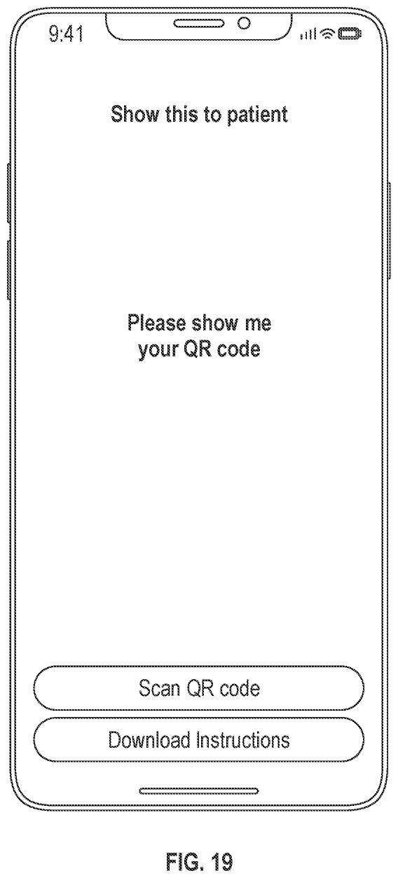

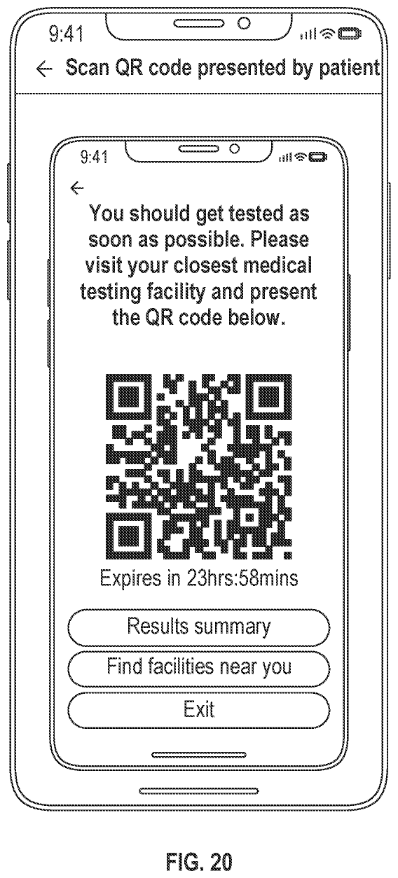

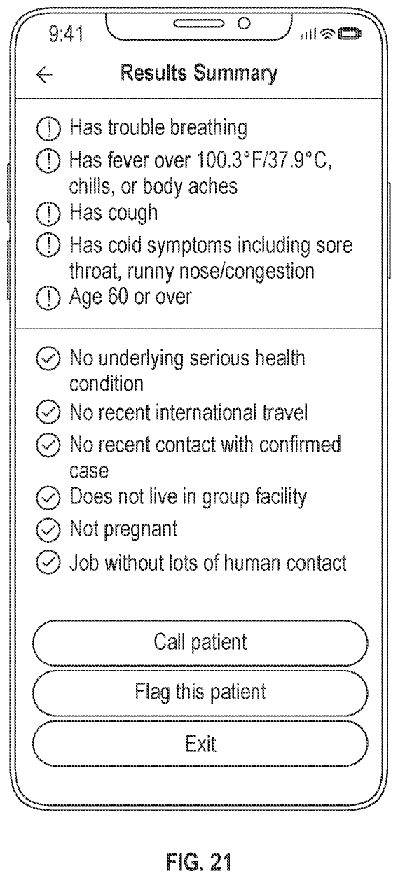

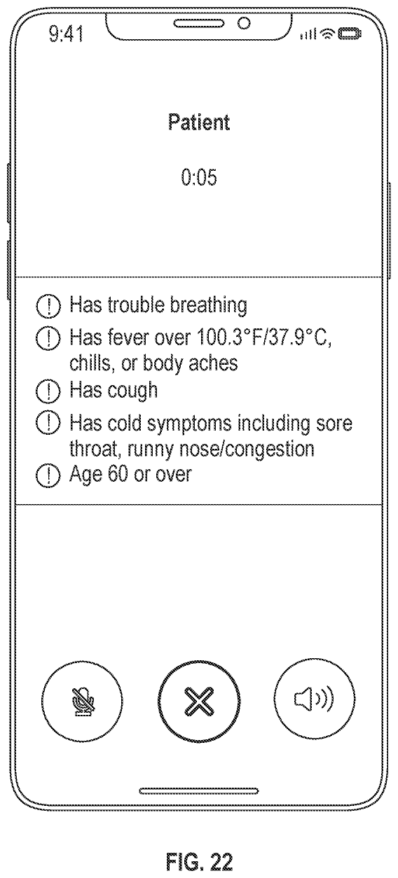

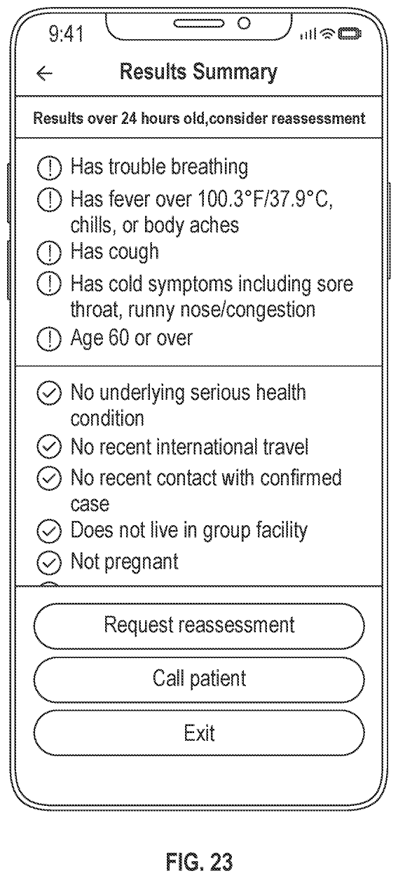

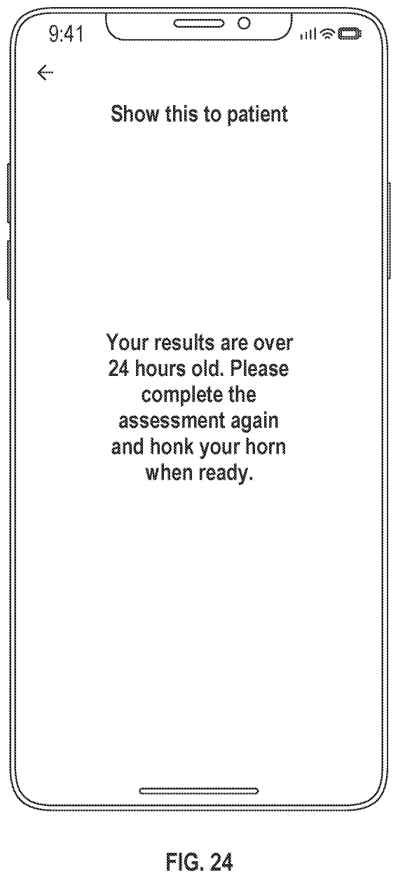

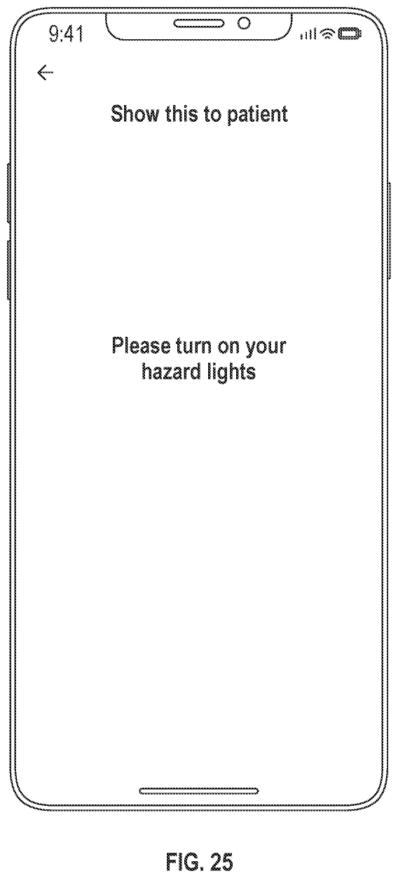

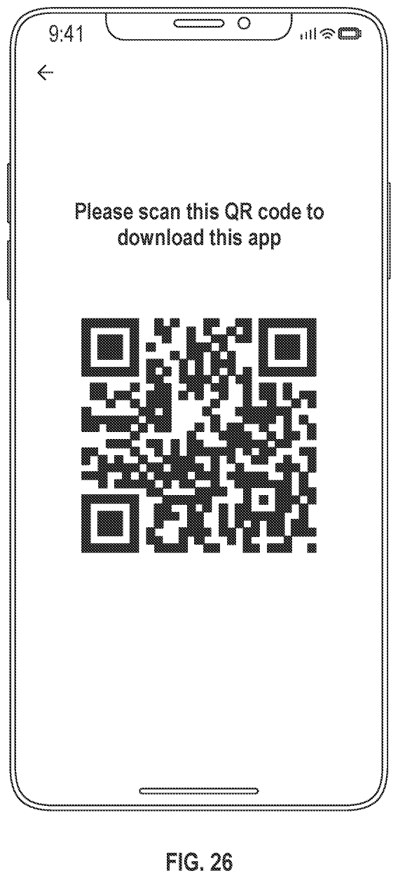

FIGS. 16-26 are screenshots illustrating some screens of an example GUI for contactless healthcare screening, as presented by a device (e.g., a mobile device, such as a smartphone or tablet) of healthcare worker to facilitate administration of a healthcare screening for a patient, according to some example embodiments.

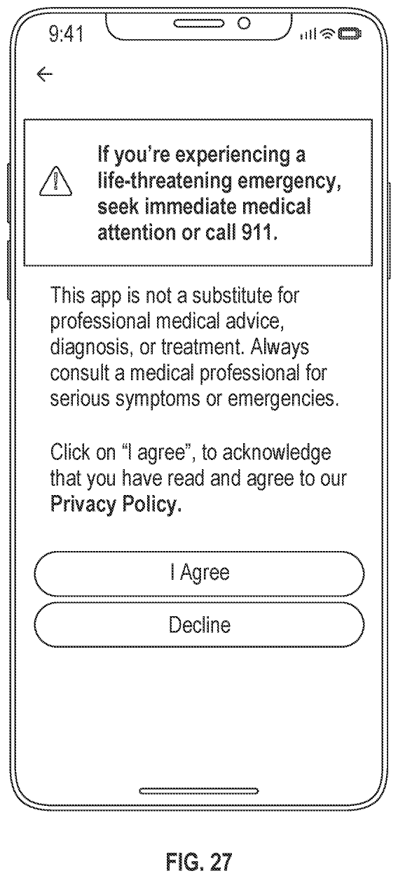

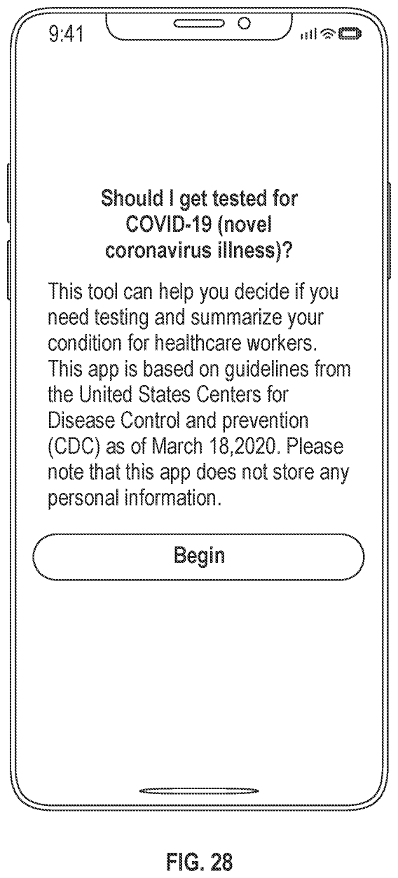

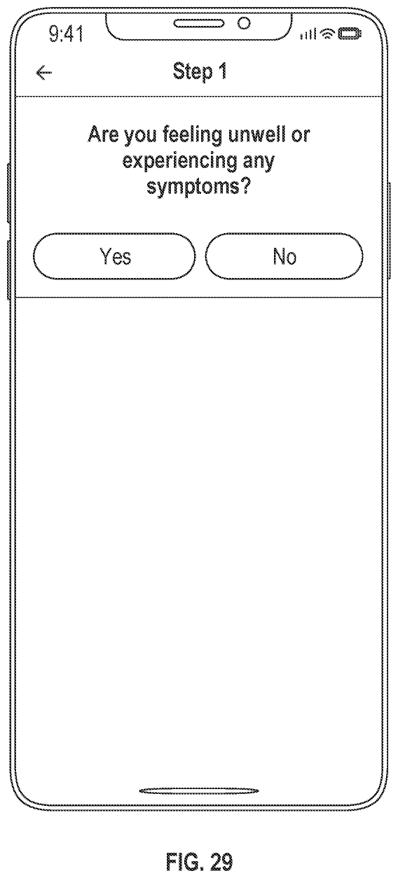

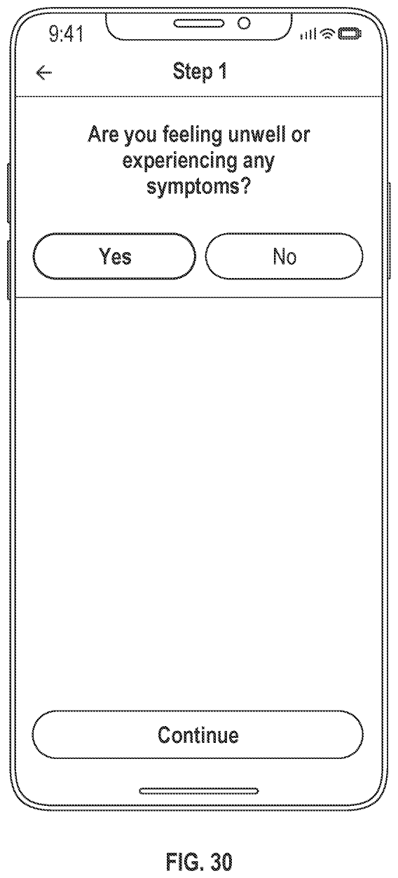

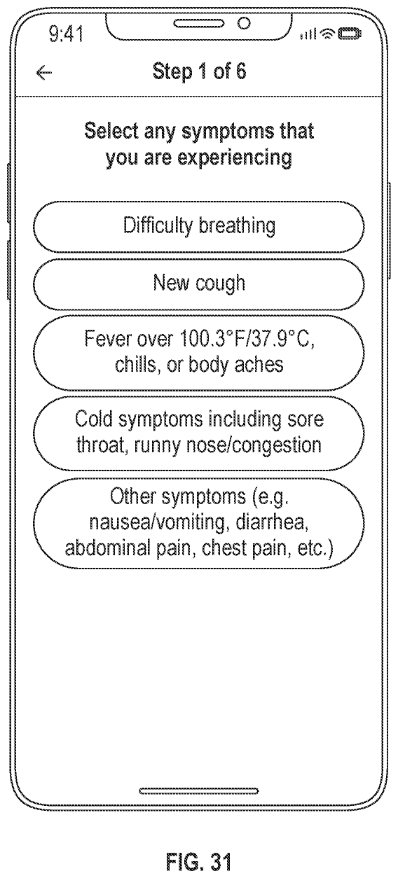

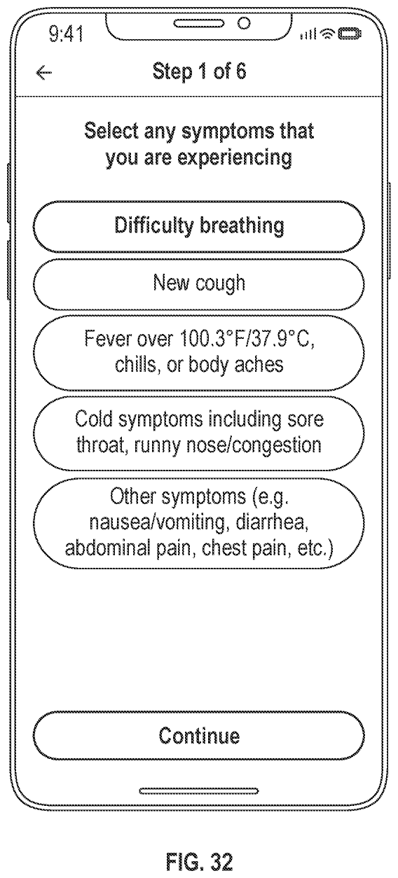

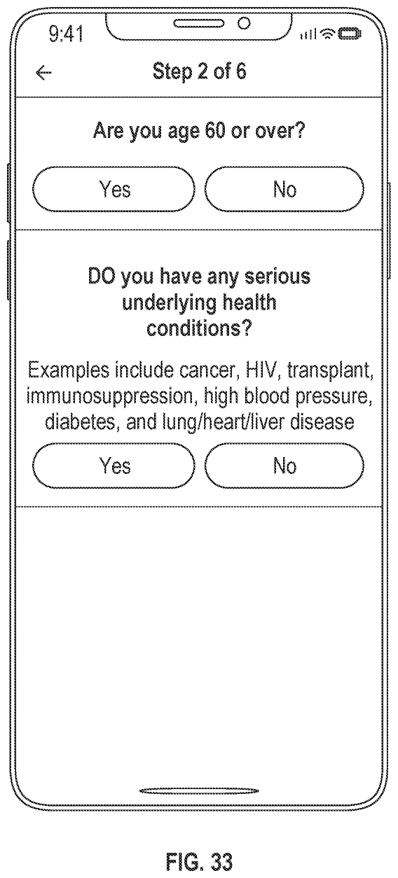

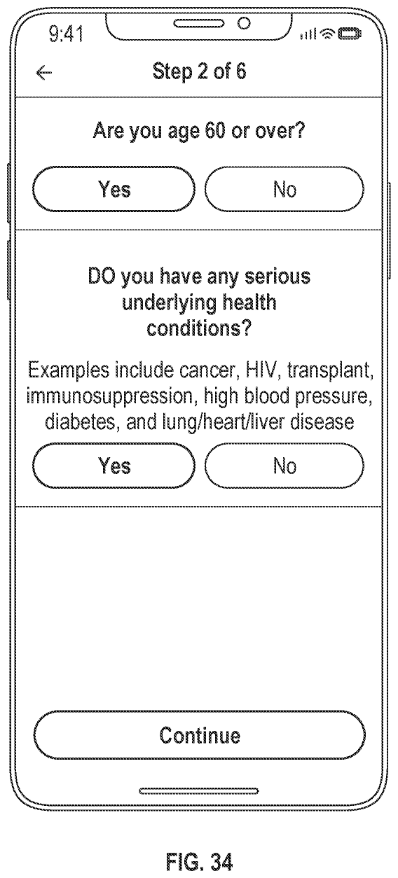

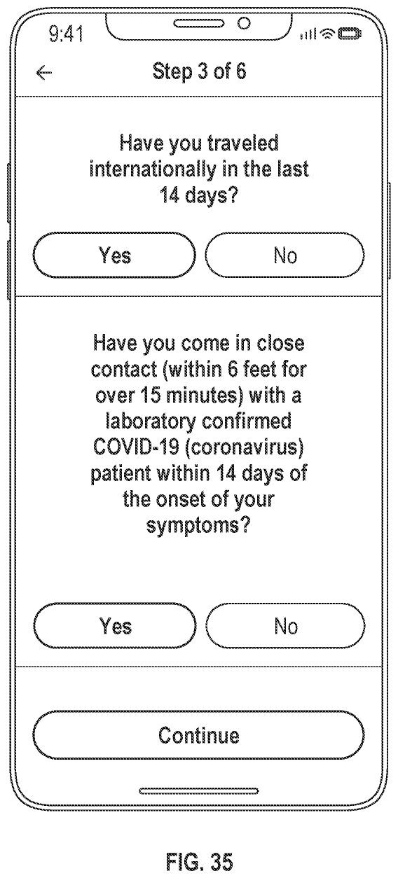

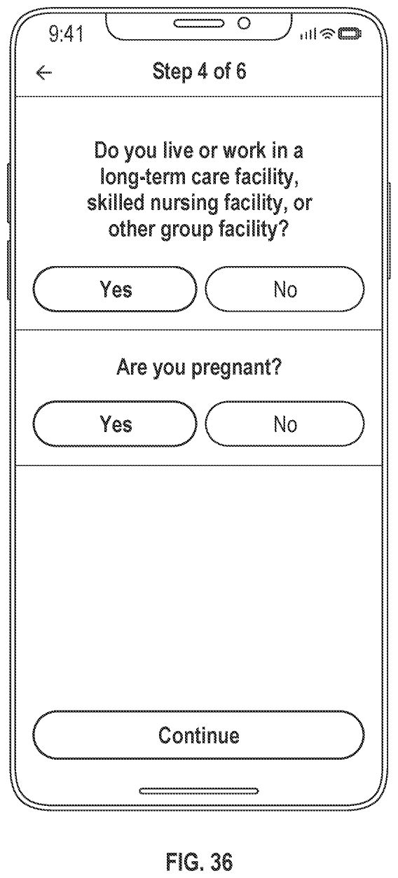

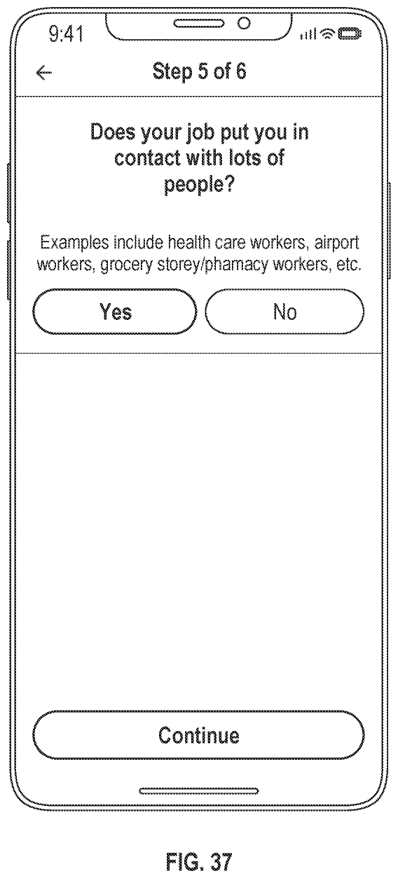

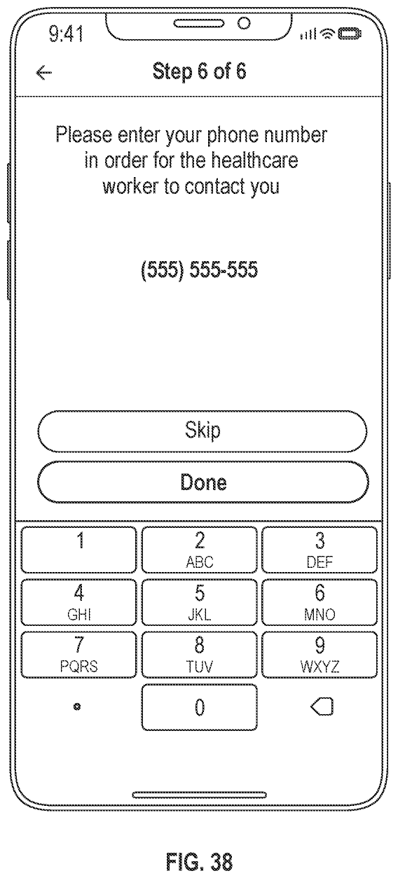

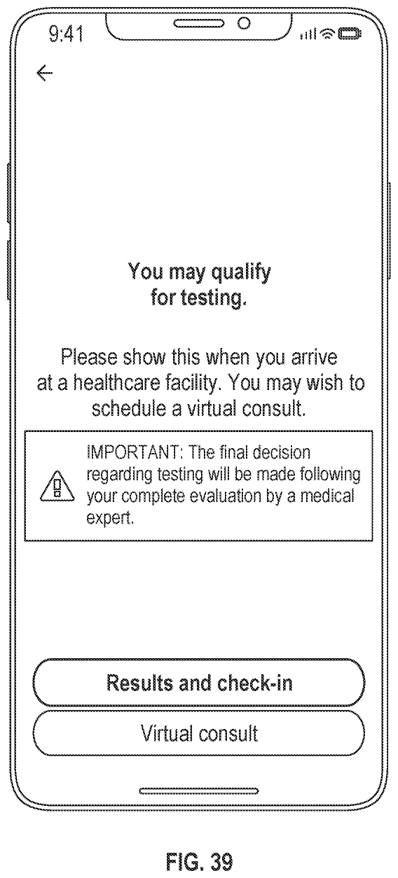

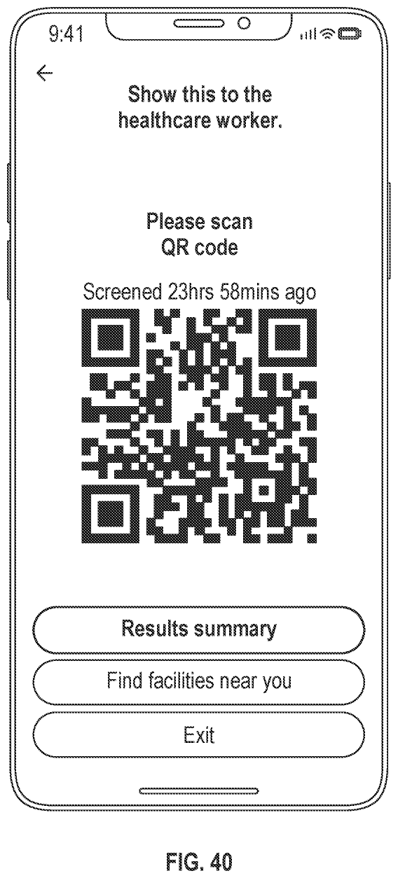

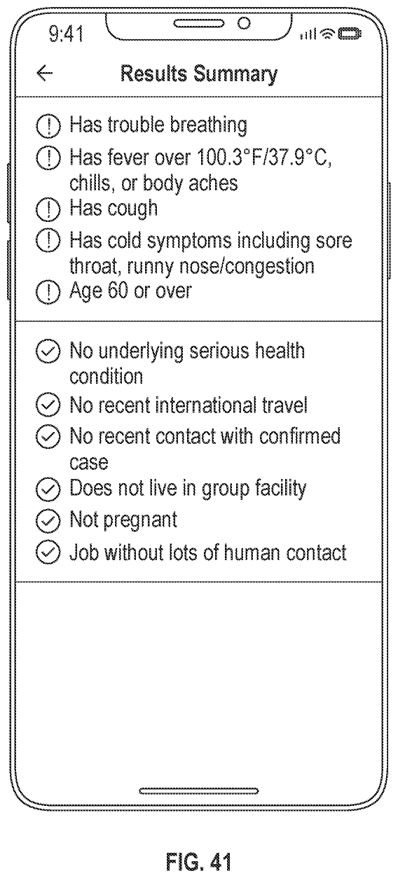

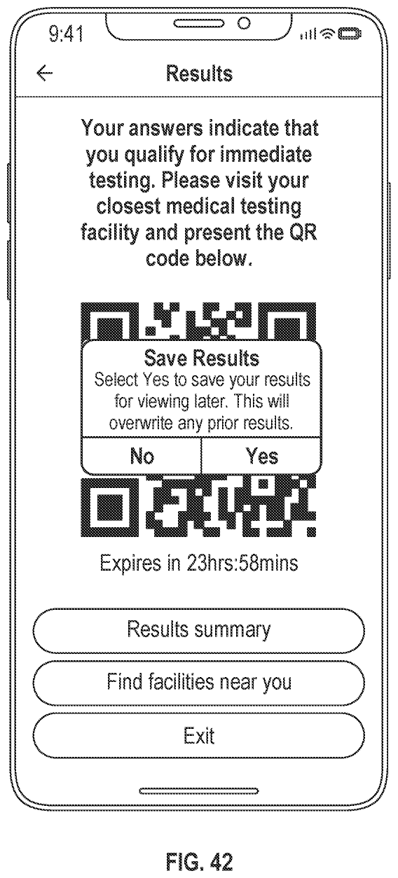

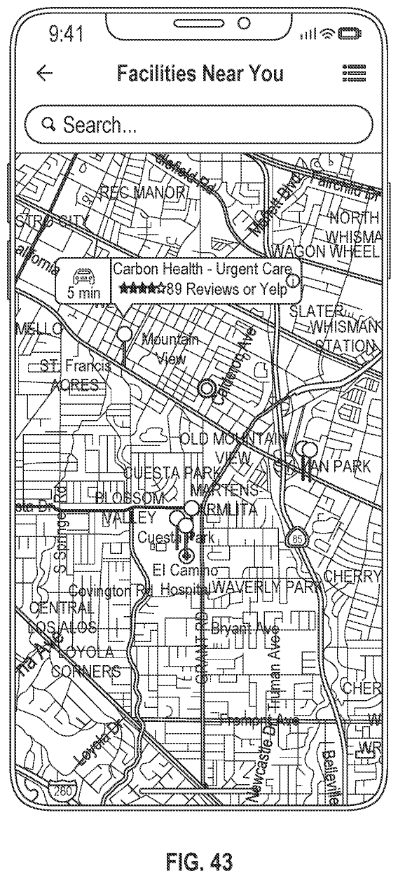

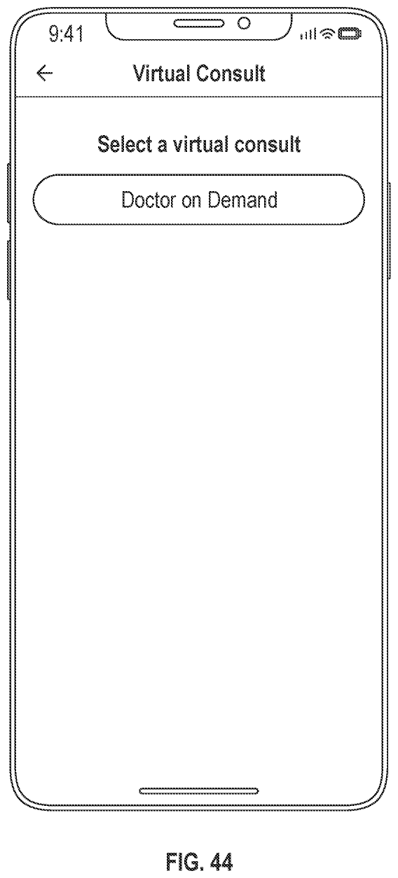

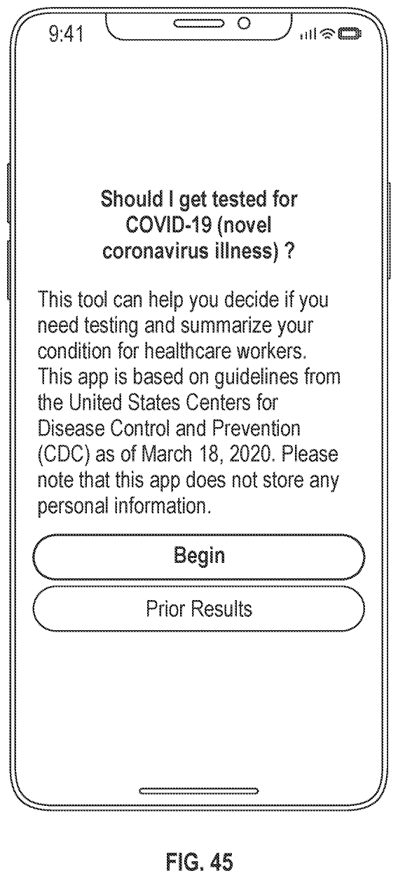

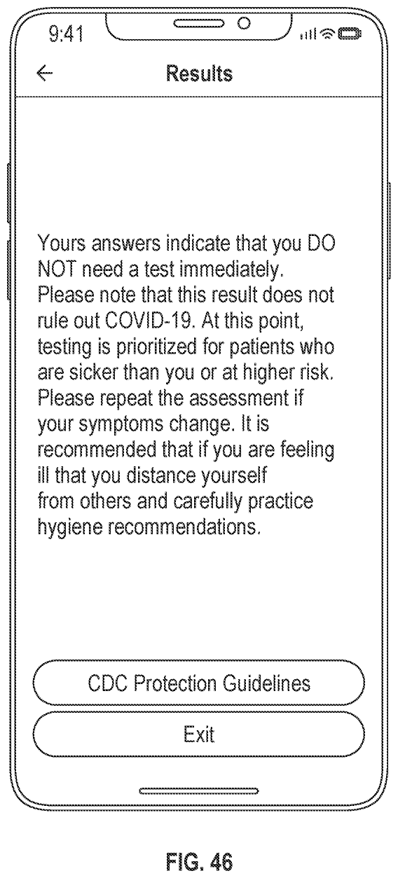

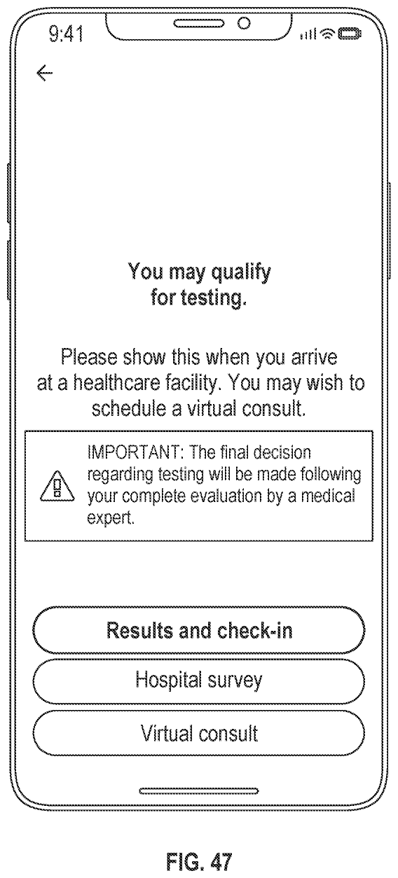

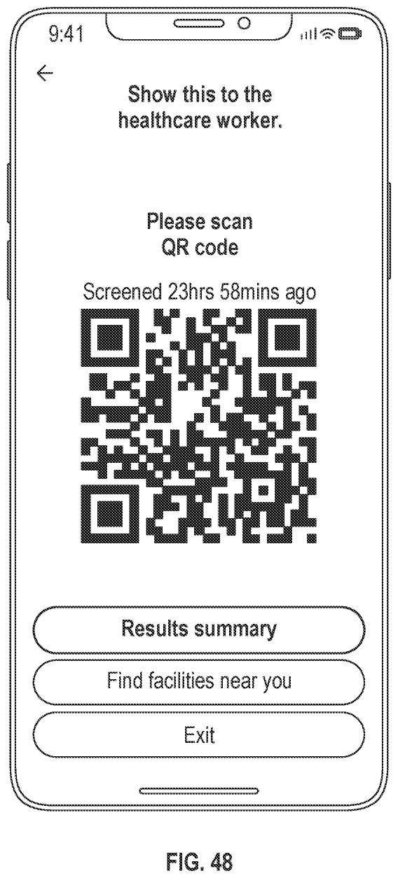

FIGS. 27-48 are screenshots illustrating some screens of an example GUI for contactless healthcare screening, as presented by a device (e.g., a mobile device, such as a smartphone or smartwatch) of a patient to facilitate participation in a healthcare screening of the patient, according to some example embodiments.

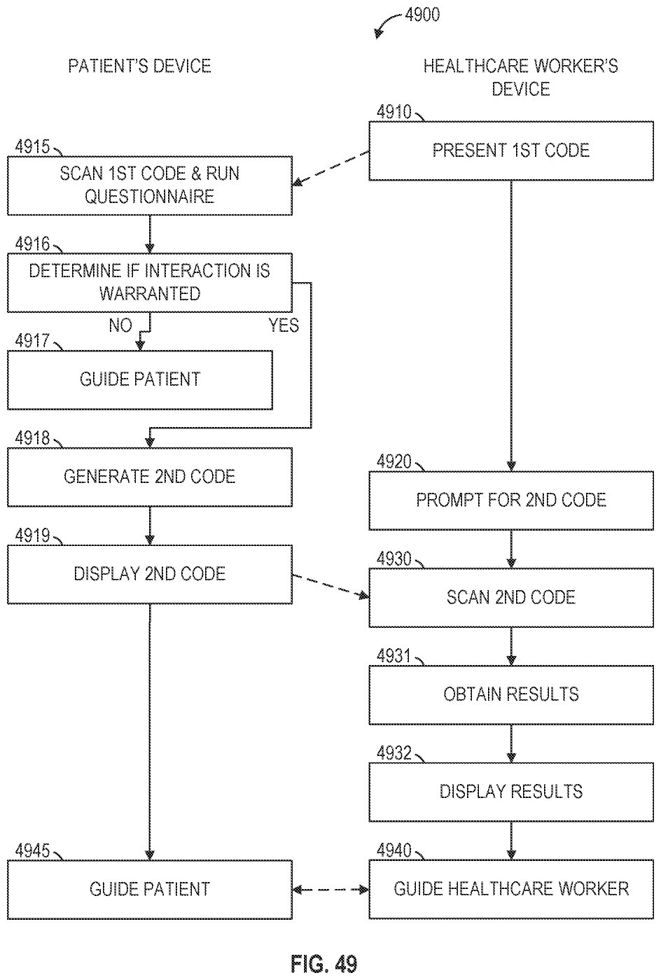

FIG. 49 is a flowchart illustrating operations of a first mobile device of a patient and a second mobile device of a healthcare worker, in performing a method 4900 of contactless healthcare screening, according to some example embodiments. Operations in the method 4900 may be performed using one or more processors (e.g., microprocessors or other hardware processors) included in the mobile device. As shown in FIG. 49, the method 4900 includes operations 4910, 4915, 4916, 4917, 4918, 4919, 4920, 4930, 4931, 4932, 4940, and 4945.

In operation 4910, a first QR code is presented to the patient (e.g., upon or shortly after the patient arrives to participate in contactless healthcare screening). The first QR code may be presented by a display screen (e.g., by the second mobile device of the healthcare worker, by a mounted display screen of a healthcare facility, by a sign board, or by any suitable combination thereof). In some example embodiments, instructions to the patient accompany the first QR code and prompt the patient to scan the first QR code. In support of contactless healthcare screening, the patient may be separated or otherwise pathogenically isolated from the healthcare worker (e.g., by one or more barriers, including transparent barriers, such as automotive glass barriers).

For example, such instructions may prompt the patient to scan the first QR code with the first mobile device of the patient (e.g., by pointing the camera of the first mobile device at the first QR code) to download a mobile app.

The instructions may additionally provide a link (e.g., a uniform resource locator (URL) to a webpage configured to administer the healthcare screening, such as via an online questionnaire).

In operation 4915, the first mobile device of the patient downloads the mobile app, if not already installed, and launches the mobile app. Operating under control of the launched mobile app, the first mobile device of the patient (e.g., via a patient GUI of a mobile app) then administers contactless healthcare screening (e.g., by administering one or more questions of a healthcare screening questionnaire) to the patient, who at this point may continue to be separated or otherwise pathogenically isolated from the healthcare worker (e.g., by one or more barriers).

In operation 4916, the first mobile device of the patient determines, based on some or all of the patient's responses to the questions asked in the administered healthcare screening questionnaire, whether an interaction (e.g., an interaction without the previous separation or other pathogenic isolation) with a healthcare worker is warranted. If not warranted, in operation 4917, the first mobile device of the patient (e.g., via the patient GUI of mobile app) guides the patient through appropriate actions (e.g., self-discharge, self-care, monitoring of symptoms at home, procurement of over-the-counter medication, etc.)

However, if the interaction with a healthcare worker is warranted, then in operation 4918, the first mobile device of the patient generates a second QR code that encodes some or all of the patient's questionnaire responses, one or more conclusions drawn from the patient's questionnaire responses, or both, as results of the contactless healthcare screening that was administered in operation 4915.

In operation 4919, the first mobile device of the patient (e.g., via the patient GUI of the mobile app) displays the generated second QR code and prompts the patient to show the generated second QR code to a healthcare worker. In some example embodiments of the patient GUI, a button or other control element is operable to display some or all of the information encoded in the second QR code. In certain example embodiments of the patient GUI, a button (e.g., a further button) or other control element (e.g., a further control element) is operable to display of one or more locations of healthcare facilities nearby. Additionally, or alternatively, in operation 4920, the second mobile device of the healthcare worker (e.g., via the healthcare worker GUI of the mobile app) prompts the healthcare worker to request that the patient present the second QR code, such as by displaying a prompt meant for the patient and asking the patient to present the second QR code. The prompt for the patient may be displayed along with instructions that the healthcare worker show the prompt to the patient (e.g., by showing the patient the display screen of the second mobile device of the healthcare worker).

In response to one or more prompts, the patient shows the second QR code to the healthcare worker (e.g., by showing the healthcare worker the display screen of the first mobile device of the patient). In operation 4930, the healthcare worker scans the second QR code (e.g., by pointing the camera of the second mobile device of the healthcare worker at the second QR code).

In operation 4931, having scanned the second QR code, the second mobile device of the healthcare worker obtains some or all of the results from the contactless healthcare screening that was administered in operation 4915. Such results may be obtained, for example, by decoding some or all of the second QR code, downloading one or more results from a server over a network (e.g., based on a patient identifier encoded in the second QR code), or any suitable combination thereof. Accordingly, in operation 4932, the second mobile device of the healthcare worker (e.g., via the healthcare worker GUI of the mobile app) displays one or more of the patient's responses to the healthcare screening questionnaire that was administered in operation 4915. The responses may be displayed along with elapsed time since the administration of the questionnaire (e.g., to give the healthcare worker an indication of a degree to which the responses are current or likely to have changed).

In some example embodiments of the healthcare worker GUI, a button or other control element is operable to initiate a call (e.g., phone, video, voice, or any suitable combination thereof) to the first mobile device of the patient (e.g., the patient's smartphone). In certain example embodiments of the healthcare worker GUI, a button (e.g., a further button) or other control element (e.g., a further control element) is operable to request a re-administration of the healthcare screening questionnaire (e.g., to obtain current responses to replace previously obtained responses that have expired or are other deemed unreliable). In such example embodiments of the healthcare worker GUI, activation of the re-administration button causes the second mobile device of the healthcare worker to prompt the healthcare worker to request that the patient repeat the healthcare screening questionnaire, such as by displaying a prompt meant for the patient and asking the patient to repeat the healthcare screening questionnaire. The prompt for the patient may be displayed along with instructions that the healthcare worker show the prompt to the patient (e.g., by showing the patient the display screen of the second mobile device of the healthcare worker).

In operation 4940, the second mobile device (e.g., via the healthcare worker GUI of the mobile app) guides the healthcare worker through his or her role in any appropriate further procedures, such as administering a healthcare test (e.g., including collection of a biological sample), administering a more refined healthcare screening questionnaire (e.g., with or without resuming the separation or other pathogenic isolation of the patient from the healthcare worker), initiating a virtual consultation (e.g., with the healthcare worker or with other healthcare personnel, such as a doctor on duty), referring the patient to another healthcare facility (e.g., a laboratory), scheduling the patient for an in-person medical appointment, or any suitable combination thereof. In operation 4945, the first mobile device of the patient (e.g., via the patient GUI of the mobile app) guides the patient through his or her role in any appropriate further procedures, in a contemporaneous and complementary manner that corresponds to performance of operation 4940 by the second mobile device of the healthcare worker.

According to various example embodiments, one or more of the methodologies described herein may facilitate contactless healthcare screening. Moreover, one or more of the methodologies described herein may facilitate administration of a questionnaire to a patient while maintaining separation or other pathogenic isolation of the patient from a healthcare worker administering the questionnaire. Hence, one or more of the methodologies described herein may facilitate a reduction in the risk of pathogen transmission, as well as increased public confidence in healthcare facilities and healthcare procedures, compared to capabilities of pre-existing systems and methods.

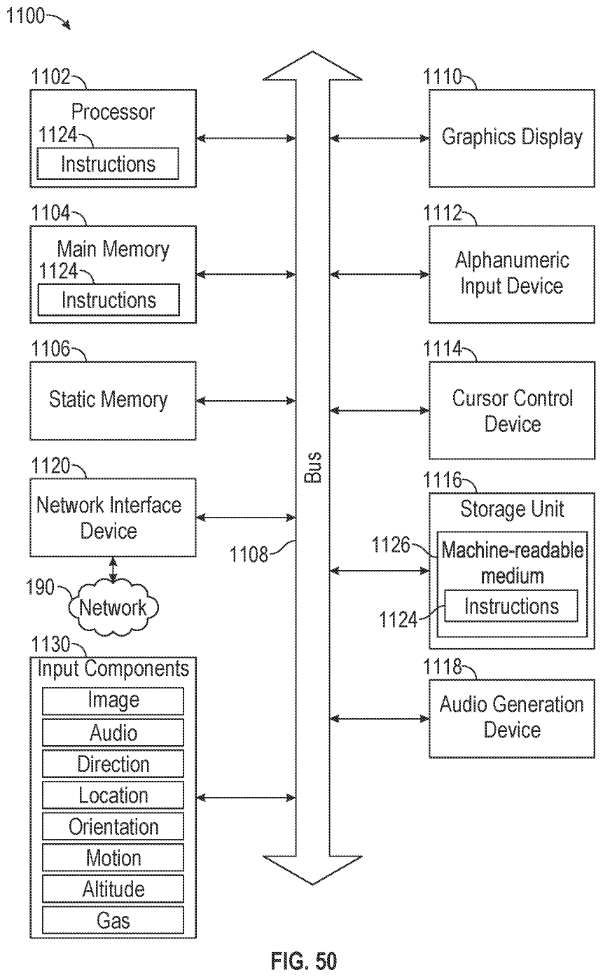

FIG. 50 is a block diagram illustrating components of a machine 1100, according to some example embodiments, able to read instructions 1124 from a machine-readable medium 1122 (e.g., a non-transitory machine-readable medium, a machine-readable storage medium, a computer-readable storage medium, or any suitable combination thereof) and perform any one or more of the methodologies discussed herein, in whole or in part. Specifically, FIG. 50 shows the machine 1100 in the example form of a computer system (e.g., a computer) within which the instructions 1124 (e.g., software, a program, an application, an applet, an app, or other executable code) for causing the machine 1100 to perform any one or more of the methodologies discussed herein may be executed, in whole or in part.

In alternative embodiments, the machine 1100 operates as a standalone device or may be communicatively coupled (e.g., networked) to other machines. In a networked deployment, the machine 1100 may operate in the capacity of a server machine or a client machine in a server-client network environment, or as a peer machine in a distributed (e.g., peer-to-peer) network environment. The machine 1100 may be a server computer, a client computer, a personal computer (PC), a tablet computer, a laptop computer, a netbook, a cellular telephone, a smart phone, a set-top box (STB), a personal digital assistant (PDA), a web appliance, a network router, a network switch, a network bridge, or any machine capable of executing the instructions 1124, sequentially or otherwise, that specify actions to be taken by that machine. Further, while only a single machine is illustrated, the term “machine” shall also be taken to include any collection of machines that individually or jointly execute the instructions 1124 to perform all or part of any one or more of the methodologies discussed herein.

The machine 1100 includes a processor 1102 (e.g., one or more central processing units (CPUs), one or more graphics processing units (GPUs), one or more digital signal processors (DSPs), one or more application specific integrated circuits (ASICs), one or more radio-frequency integrated circuits (RFICs), or any suitable combination thereof), a main memory 1104, and a static memory 1106, which are configured to communicate with each other via a bus 1108. The processor 1102 contains solid-state digital microcircuits (e.g., electronic, optical, or both) that are configurable, temporarily or permanently, by some or all of the instructions 1124 such that the processor 1102 is configurable to perform any one or more of the methodologies described herein, in whole or in part. For example, a set of one or more microcircuits of the processor 1102 may be configurable to execute one or more modules (e.g., software modules) described herein. In some example embodiments, the processor 1102 is a multicore CPU (e.g., a dual-core CPU, a quad-core CPU, an 8-core CPU, or a 128-core CPU) within which each of multiple cores behaves as a separate processor that is able to perform any one or more of the methodologies discussed herein, in whole or in part. Although the beneficial effects described herein may be provided by the machine 1100 with at least the processor 1102, these same beneficial effects may be provided by a different kind of machine that contains no processors (e.g., a purely mechanical system, a purely hydraulic system, or a hybrid mechanical-hydraulic system), if such a processor-less machine is configured to perform one or more of the methodologies described herein.

The machine 1100 may further include a graphics display 1110 (e.g., a plasma display panel (PDP), a light emitting diode (LED) display, a liquid crystal display (LCD), a projector, a cathode ray tube (CRT), or any other display capable of displaying graphics or video). The machine 1100 may also include an alphanumeric input device 1112 (e.g., a keyboard or keypad), a pointer input device 1114 (e.g., a mouse, a touchpad, a touchscreen, a trackball, a joystick, a stylus, a motion sensor, an eye tracking device, a data glove, or other pointing instrument), a data storage 1116, an audio generation device 1118 (e.g., a sound card, an amplifier, a speaker, a headphone jack, or any suitable combination thereof), and a network interface device 1120.

The data storage 1116 (e.g., a data storage device) includes the machine-readable medium 1122 (e.g., a tangible and non-transitory machine-readable storage medium) on which are stored the instructions 1124 embodying any one or more of the methodologies or functions described herein. The instructions 1124 may also reside, completely or at least partially, within the main memory 1104, within the static memory 1106, within the processor 1102 (e.g., within the processor's cache memory), or any suitable combination thereof, before or during execution thereof by the machine 1100. Accordingly, the main memory 1104, the static memory 1106, and the processor 1102 may be considered machine-readable media (e.g., tangible and non-transitory machine-readable media). The instructions 1124 may be transmitted or received over the network 190 via the network interface device 1120. For example, the network interface device 1120 may communicate the instructions 1124 using any one or more transfer protocols (e.g., hypertext transfer protocol (HTTP)).

In some example embodiments, the machine 1100 may be a portable computing device (e.g., a smart phone, a tablet computer, or a wearable device) and may have one or more additional input components 1130 (e.g., sensors or gauges). Examples of such input components 1130 include an image input component (e.g., one or more cameras), an audio input component (e.g., one or more microphones), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), a temperature input component (e.g., a thermometer), and a gas detection component (e.g., a gas sensor). Input data gathered by any one or more of these input components 1130 may be accessible and available for use by any of the modules described herein (e.g., with suitable privacy notifications and protections for personally identifiable information (PII), such as opt-in consent or opt-out consent, implemented in accordance with user preference, applicable regulations, or any suitable combination thereof).

As used herein, the term “memory” refers to a machine-readable medium able to store data temporarily or permanently and may be taken to include, but not be limited to, random-access memory (RAM), read-only memory (ROM), buffer memory, flash memory, and cache memory. While the machine-readable medium 1122 is shown in an example embodiment to be a single medium, the term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of carrying (e.g., storing or communicating) the instructions 1124 for execution by the machine 1100, such that the instructions 1124, when executed by one or more processors of the machine 1100 (e.g., processor 1102), cause the machine 1100 to perform any one or more of the methodologies described herein, in whole or in part. Accordingly, a “machine-readable medium” refers to a single storage apparatus or device, as well as cloud-based storage systems or storage networks that include multiple storage apparatus or devices. The term “machine-readable medium” shall accordingly be taken to include, but not be limited to, one or more tangible and non-transitory data repositories (e.g., data volumes) in the example form of a solid-state memory chip, an optical disc, a magnetic disc, or any suitable combination thereof.

A “non-transitory” machine-readable medium, as used herein, specifically excludes propagating signals per se. According to various example embodiments, the instructions 1124 for execution by the machine 1100 can be communicated via a carrier medium (e.g., a machine-readable carrier medium).

Examples of such a carrier medium include a non-transient carrier medium (e.g., a non-transitory machine-readable storage medium, such as a solid-state memory that is physically movable from one place to another place) and a transient carrier medium (e.g., a carrier wave or other propagating signal that communicates the instructions 1124).

Certain example embodiments are described herein as including modules. Modules may constitute software modules (e.g., code stored or otherwise embodied in a machine-readable medium or in a transmission medium), hardware modules, or any suitable combination thereof. A “hardware module” is a tangible (e.g., non-transitory) physical component (e.g., a set of one or more processors) capable of performing certain operations and may be configured or arranged in a certain physical manner. In various example embodiments, one or more computer systems or one or more hardware modules thereof may be configured by software (e.g., an application or portion thereof) as a hardware module that operates to perform operations described herein for that module.

In some example embodiments, a hardware module may be implemented mechanically, electronically, hydraulically, or any suitable combination thereof. For example, a hardware module may include dedicated circuitry or logic that is permanently configured to perform certain operations. A hardware module may be or include a special-purpose processor, such as a field programmable gate array (FPGA) or an ASIC. A hardware module may also include programmable logic or circuitry that is temporarily configured by software to perform certain operations. As an example, a hardware module may include software encompassed within a CPU or other programmable processor. It will be appreciated that the decision to implement a hardware module mechanically, hydraulically, in dedicated and permanently configured circuitry, or in temporarily configured circuitry (e.g., configured by software) may be driven by cost and time considerations.

Accordingly, the phrase “hardware module” should be understood to encompass a tangible entity that may be physically constructed, permanently configured (e.g., hardwired), or temporarily configured (e.g., programmed) to operate in a certain manner or to perform certain operations described herein. Furthermore, as used herein, the phrase “hardware-implemented module” refers to a hardware module. Considering example embodiments in which hardware modules are temporarily configured (e.g., programmed), each of the hardware modules need not be configured or instantiated at any one instance in time. For example, where a hardware module includes a CPU configured by software to become a special-purpose processor, the CPU may be configured as respectively different special-purpose processors (e.g., each included in a different hardware module) at different times. Software (e.g., a software module) may accordingly configure one or more processors, for example, to become or otherwise constitute a particular hardware module at one instance of time and to become or otherwise constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory (e.g., a memory device) to which it is communicatively coupled. A further hardware module may then, at a later time, access the memory to retrieve and process the stored output. Hardware modules may also initiate communications with input or output devices, and can operate on a resource (e.g., a collection of information from a computing resource).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module in which the hardware includes one or more processors. Accordingly, the operations described herein may be at least partially processor-implemented, hardware-implemented, or both, since a processor is an example of hardware, and at least some operations within any one or more of the methods discussed herein may be performed by one or more processor-implemented modules, hardware-implemented modules, or any suitable combination thereof.

Moreover, such one or more processors may perform operations in a “cloud computing” environment or as a service (e.g., within a “software as a service” (SaaS) implementation). For example, at least some operations within any one or more of the methods discussed herein may be performed by a group of computers (e.g., as examples of machines that include processors), with these operations being accessible via a network (e.g., the Internet) and via one or more appropriate interfaces (e.g., an application program interface (API)). The performance of certain operations may be distributed among the one or more processors, whether residing only within a single machine or deployed across a number of machines. In sonic example embodiments, the one or more processors or hardware modules (e.g., processor-implemented modules) may be located in a single geographic location (e.g., within a home environment, an office environment, or a server farm). In other example embodiments, the one or more processors or hardware modules may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and their functionality presented as separate components and functions in example configurations may be implemented as a combined structure or component with combined functions. Similarly, structures and functionality presented as a single component may be implemented as separate components and functions. These and other variations, modifications, additions, and improvements fall within the scope of the subject matter herein.

Some portions of the subject matter discussed herein may be presented in terms of algorithms or symbolic representations of operations on data stored as bits or binary digital signals within a memory (e.g., a computer memory or other machine memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “accessing,” “processing,” “detecting,” “computing,” “calculating,” “determining,” “generating,” “presenting,” “displaying,” or the like refer to actions or processes performable by a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The following enumerated descriptions describe various examples of methods, machine-readable media, and systems (e.g., machines, devices, or other apparatus) discussed herein. It should be noted that one or more features of an example, taken in isolation or combination, should be considered within the disclosure of this application.

A first example provides a method comprising:
administering, by one or more processors of a patient device (e.g., a first mobile device), a questionnaire to a user of the patient device in response to installation and launch of a mobile app obtainable by scanning a first optically readable code that encodes instructions to obtain the mobile app;
generating, by the one or more processors of the patient device and in response to completion of the administering of the questionnaire to the user of the patient device, a second optically readable code that encodes a result of the questionnaire administered to the user of the patient device; and
causing, by the one or more processors of the patient device, presentation of the generated second optically readable code that encodes the result of the questionnaire administered to the user of the patient device, the presented second optically readable code being scannable by a worker device (e.g., a second mobile device) to obtain, at the worker device, the encoded result of the questionnaire administered to the user of the patient device.

A second example provides a method according to the first example, wherein:
the scanning of the first optically readable code that encodes instructions to obtain the mobile app is initiated by the patient device in response to the first optically readable code being presented, to the patient device, by a mounted display screen of a facility.

A third example provides a method according to the first example or the second example, wherein:
the scanning of the first optically readable code that encodes instructions to obtain the mobile app is initiated by the patient device in response to the first optically readable code being presented, to the patient device, by a mobile display screen of the worker device.

A fourth example provides a method according to any of the first through third examples, wherein:
the user of the patient device is a first user; and
the method further comprises:
determining whether an in-person interaction between the first user of the patient device and a second user of the worker device is to occur.

A fifth example provides a method according to the fourth example, wherein:
the determining of whether the in-person interaction between the first user of the patient device and the second user of the worker device is to occur includes determining that the in-person interaction between the first user of the patient device and the second user of the worker device is to occur; and
the generating of the second optically readable code that encodes the result of the questionnaire administered to the first user of the patient device is in response to the determining that the in-person interaction between the first user of the patient device and the second user of the worker device is to occur.

A sixth example provides a method comprising:
scanning, by one or more processors of a worker device (e.g., a first mobile device), a first optically readable code that encodes a result of a questionnaire administered to a first user of a patient device (e.g., a second mobile device) in response to installation and launch of a mobile app obtainable by scanning a second optically readable code that encodes instructions to obtain the mobile app;
obtaining, by the one or more processors of the worker device and based on the scanned first optically readable code, the result of the questionnaire administered to the first user of the patient device; and
causing, by the one or more processors of the worker device and to a second user of the worker device, presentation of the obtained result of the questionnaire administered to the first user of the patient device.

A seventh example provides a method according to the sixth example, further comprising:
causing presentation of the second optically readable code that encodes the instructions to obtain the mobile app obtainable by the scanning of the second optically readable code, the mobile app configuring the patient device to administer the questionnaire to the first user of the patient device.

An eighth example provides a method according to the seventh example, wherein:
the scanning of the first optically readable code that encodes the result of the questionnaire administered to the first user of the patient device is in response to presentation of the first optically readable code to the worker device by the patient device.

A ninth example provides a method according to the eighth example, wherein:
the presentation of the first optically readable code that encodes the result of the questionnaire administered to the first user of the patient device is in response to presentation of a prompt by the worker device that the second user of the worker device request that the first user of the patient device present the first optically readable code to the worker device.

A tenth example provides a method according to any of the sixth through ninth examples, further comprising:
causing presentation of an instruction, to the second user of the worker device, that the second user of the worker device guide the first user of the patient device in a procedure.

An eleventh example provides a machine-readable medium (e.g., a non-transitory machine-readable storage medium) comprising instructions that, when executed by one or more processors of a patient device, cause the patient device to perform operations comprising:
administering a questionnaire to a user of the patient device in response to installation and launch of a mobile app obtainable by scanning a first optically readable code that encodes instructions to obtain the mobile app;
generating, in response to completion of the administering of the questionnaire to the user of the patient device, a second optically readable code that encodes a result of the questionnaire administered to the user of the patient device; and
causing presentation of the generated second optically readable code that encodes the result of the questionnaire administered to the user of the patient device, the presented second optically readable code being scannable by a worker device to obtain, at the worker device, the encoded result of the questionnaire administered to the user of the patient device.

A twelfth example provides a machine-readable medium according to the eleventh example, wherein:
the scanning of the first optically readable code that encodes instructions to obtain the mobile app is initiated by the patient device in response to the first optically readable code being presented, to the patient device, by a mobile display screen of the worker device.

A thirteenth example provides a machine-readable medium according to the eleventh example or the twelfth example, wherein:
the user of the patient device is a first user; and
the operations further comprise:
determining whether an in-person interaction between the first user of the patient device and a second user of the worker device is to occur.

A fourteenth example provides a machine-readable medium (e.g., a non-transitory machine-readable storage medium) comprising instructions that, when executed by one or more processors of a worker device, cause the worker device to perform operations comprising:
scanning a first optically readable code that encodes a result of a questionnaire administered to a first user of a patient device in response to installation and launch of a mobile app obtainable by scanning a second optically readable code that encodes instructions to obtain the mobile app;
obtaining, based on the scanned first optically readable code, the result of the questionnaire administered to the first user of the patient device; and
causing, to a second user of the worker device, presentation of the obtained result of the questionnaire administered to the first user of the patient device.

A fifteenth example provides a machine-readable medium according to the fourteenth example, wherein the operations further comprise:
causing presentation of the second optically readable code that encodes the instructions to obtain the mobile app obtainable by the scanning of the second optically readable code, the mobile app configuring the patient device to administer the questionnaire to the first user of the patient device.

A sixteenth example provides a machine-readable medium according to the fifteenth example, wherein:
the scanning of the first optically readable code that encodes the result of the questionnaire administered to the first user of the patient device is in response to presentation of the first optically readable code to the worker device by the patient device.

A seventeenth example provides a system (e.g., a computer system) comprising:
one or more processors; and
a memory storing instructions that, when executed by at least one processor among the one or more processors, cause the system to perform operations comprising:
administering a questionnaire to a user of a patient device in response to installation and launch of a mobile app obtainable by scanning a first optically readable code that encodes instructions to obtain the mobile app;
generating, in response to completion of the administering of the questionnaire to the user of the patient device, a second optically readable code that encodes a result of the questionnaire administered to the user of the patient device; and
causing presentation of the generated second optically readable code that encodes the result of the questionnaire administered to the user of the patient device, the presented second optically readable code being scannable by a worker device to obtain, at the worker device, the encoded result of the questionnaire administered to the user of the patient device.

An eighteenth example provides a system according to the seventeenth example, wherein:
the scanning of the first optically readable code that encodes instructions to obtain the mobile app is initiated by the patient device in response to the first optically readable code being presented, to the patient device, by a mobile display screen of the worker device.

A nineteenth example provides a system (e.g., a computer system) comprising:
one or more processors; and
a memory storing instructions that, when executed by at least one processor among the one or more processors, cause the system to perform operations comprising:
scanning a first optically readable code that encodes a result of a questionnaire administered to a first user of a patient device in response to installation and launch of a mobile app obtainable by scanning a second optically readable code that encodes instructions to obtain the mobile app;
obtaining, based on the scanned first optically readable code, the result of the questionnaire administered to the first user of the patient device; and
causing, to a second user of a worker device, presentation of the obtained result of the questionnaire administered to the first user of the patient device.

A twentieth example provides a system according to the nineteenth example, wherein the operations further comprise:
causing presentation of the second optically readable code that encodes the instructions to obtain the mobile app obtainable by the scanning of the second optically readable code, the mobile app configuring the patient device to administer the questionnaire to the first user of the patient device.

A twenty-first example provides a carrier medium carrying machine-readable instructions for controlling a machine to carry out the operations (e.g., method operations) performed in any one of the previously described examples.

What is claimed is:

1. A method comprising:
administering, by one or more processors of a patient device, a questionnaire to a user of the patient device in response to installation and launch of a mobile app obtainable by scanning a first optically readable code that encodes instructions to obtain the mobile app;
generating, by the one or more processors of the patient device and in response to completion of the administering of the questionnaire to the user of the patient device, a second optically readable code that encodes a result of the questionnaire administered to the user of the patient device; and
causing, by the one or more processors of the patient device, presentation of the generated second optically readable code that encodes the result of the questionnaire administered to the user of the patient device, the presented second optically readable code being scannable by a worker device to obtain, at the worker device, the encoded result of the questionnaire administered to the user of the patient device.

2. The method of claim 1, wherein:
the scanning of the first optically readable code that encodes instructions to obtain the mobile app is initiated by the patient device in response to the first optically readable code being presented, to the patient device, by a mounted display screen of a facility.

3. The method of claim 1, wherein:
the scanning of the first optically readable code that encodes instructions to obtain the mobile app is initiated by the patient device in response to the first optically readable code being presented, to the patient device, by a mobile display screen of the worker device.

4. The method of claim 1, wherein:
the user of the patient device is a first user; and
the method further comprises:
determining whether an in-person interaction between the first user of the patient device and a second user of the worker device is to occur.

5. The method of claim 4, wherein:
the determining of whether the in-person interaction between the first user of the patient device and the second user of the worker device is to occur includes determining that the in-person interaction between the first user of the patient device and the second user of the worker device is to occur; and
the generating of the second optically readable code that encodes the result of the questionnaire administered to the first user of the patient device is in response to the determining that the in-person interaction between the first user of the patient device and the second user of the worker device is to occur.

6. A method comprising:
scanning, by one or more processors of a worker device, a first optically readable code that encodes a result of a questionnaire administered to a first user of a patient device in response to installation and launch of a mobile app obtainable by scanning a second optically readable code that encodes instructions to obtain the mobile app;
obtaining, by the one or more processors of the worker device and based on the scanned first optically readable code, the result of the questionnaire administered to the first user of the patient device; and
causing, by the one or more processors of the worker device and to a second user of the worker device, presentation of the obtained result of the questionnaire administered to the first user of the patient device.

7. The method of claim 6, further comprising:
causing presentation of the second optically readable code that encodes the instructions to obtain the mobile app obtainable by the scanning of the second optically readable code, the mobile app configuring the patient device to administer the questionnaire to the first user of the patient device.

8. The method of claim 7, wherein:
the scanning of the first optically readable code that encodes the result of the questionnaire administered to the first user of the patient device is in response to presentation of the first optically readable code to the worker device by the patient device.

9. The method of claim 8, wherein:
the presentation of the first optically readable code that encodes the result of the questionnaire administered to the first user of the patient device is in response to presentation of a prompt by the worker device that the second user request the first user to present the first optically readable code to the worker device.

10. The method of claim 6, further comprising:
causing presentation of an instruction, to the second user of the worker device, that the second user of the worker device guide the first user of the patient device in a procedure.

11. A non-transitory machine-readable medium comprising instructions that, when executed by one or more processors of a patient device, cause the patient device to perform operations comprising:
administering a questionnaire to a user of the patient device in response to installation and launch of a mobile app obtainable by scanning a first optically readable code that encodes instructions to obtain the mobile app;
generating, in response to completion of the administering of the questionnaire to the user of the patient device, a second optically readable code that encodes a result of the questionnaire administered to the user of the patient device; and
causing presentation of the generated second optically readable code that encodes the result of the questionnaire administered to the user of the patient device, the presented second optically readable code being scannable by a worker device to obtain, at the worker device, the encoded result of the questionnaire administered to the user of the patient device.

12. The non-transitory machine-readable medium of claim 11, wherein:
the scanning of the first optically readable code that encodes instructions to obtain the mobile app is initiated by the patient device in response to the first optically readable code being presented, to the patient device, by a mobile display screen of the worker device.

13. The non-transitory machine-readable medium of claim 11, wherein:
the user of the patient device is a first user; and
the operations further comprise:
determining whether an in-person interaction between the first user of the patient device and a second user of the worker device is to occur.

14. A non-transitory machine-readable medium comprising instructions that, when executed by one or more processors of a worker device, cause the worker device to perform operations comprising:
scanning a first optically readable code that encodes a result of a questionnaire administered to a first user of a patient device in response to installation and launch of a mobile app obtainable by scanning a second optically readable code that encodes instructions to obtain the mobile app;
obtaining, based on the scanned first optically readable code, the result of the questionnaire administered to the first user of the patient device; and
causing, to a second user of the worker device, presentation of the obtained result of the questionnaire administered to the first user of the patient device.

15. The non-transitory machine-readable medium of claim 14, wherein the operations further comprise:
causing presentation of the second optically readable code that encodes the instructions to obtain the mobile app obtainable by the scanning of the second optically readable code, the mobile app configuring the patient device to administer the questionnaire to the first user of the patient device.

16. The non-transitory machine-readable medium of claim 15, wherein:
the scanning of the first optically readable code that encodes the result of the questionnaire administered to the first user of the patient device is in response to presentation of the first optically readable code to the worker device by the patient device.

17. A system comprising:
one or more processors; and
a memory storing instructions that, when executed by at least one processor among the one or more processors, cause the system to perform operations comprising:
administering a questionnaire to a user of a patient device in response to installation and launch of a mobile app obtainable by scanning a first optically readable code that encodes instructions to obtain the mobile app;
generating, in response to completion of the administering of the questionnaire to the user of the patient device, a second optically readable code that encodes a result of the questionnaire administered to the user of the patient device; and
causing presentation of the generated second optically readable code that encodes the result of the questionnaire administered to the user of the patient device, the presented second optically readable code being scannable by a worker device to obtain, at the worker device, the encoded result of the questionnaire administered to the user of the patient device.

18. The system of claim 17, wherein:
the scanning of the first optically readable code that encodes instructions to obtain the mobile app is initiated by the patient device in response to the first optically readable code being presented, to the patient device, by a mobile display screen of the worker device.

19. A system comprising:
one or more processors; and
a memory storing instructions that, when executed by at least one processor among the one or more processors, cause the system to perform operations comprising:
scanning a first optically readable code that encodes a result of a questionnaire administered to a first user of a patient device in response to installation and launch of a mobile app obtainable by scanning a second optically readable code that encodes instructions to obtain the mobile app;
obtaining, based on the scanned first optically readable code, the result of the questionnaire administered to the first user of the patient device; and
causing, to a second user of a worker device, presentation of the Obtained result of the questionnaire administered to the first user of the patient device.

20. The system of claim 19, wherein the operations further comprise:
causing presentation of the second optically readable code that encodes the instructions to obtain the mobile app obtainable by the scanning of the second optically readable code, the mobile app configuring the patient device to administer the questionnaire to the first user of the patient device.
